(19) United States
(12) Patent Application Publication
(10) Pub. No.: US 2010/0168572 A1
(43) Pub. Date: Jul. 1, 2010

(54) Apparatus and Methods for Acoustic Monitoring of Ablation Procedures

(76) Inventors: John W. Sliwa, Los Altos Hills, CA (US); Zhenyi Ma, San Jose, CA (US); John P. Goetz, Aptos, CA (US); Stephen A. Morse, Menlo Park, CA (US)

Correspondence Address: SJM/AFD-WILEY;Legal Department, One St. Jude Medical Drive, St. Paul, MN, 55117-9913, US

(21) Appl. No.: 12/636,837

(22) Filed: Dec. 14, 2009

Related U.S. Application Data
(60) Provisional application No. 61/141,379, filed on Dec. 30, 2008.

(51) Int. Cl. A61B 8/00; A61B 18/18

(52) U.S. Cl. 600/439; 606/33

(57) ABSTRACT

A system for ablating tissue includes a catheter having an elongate body, with at least one ablation element (e.g., RF electrode) and at least one acoustic transducer located within the body's tip region. The transducer receives acoustic signals from proximate the tip region. The system also includes a monitoring unit coupled to the transducer to interpret the received acoustic signals as data regarding at least one therapeutic parameter (e.g., pre-pop detection, lesion making progress, tissue interface detection, tissue contact force, tissue contact establishment, bubble spatial distribution, bubble depth, bubble size, bubble size distribution, tissue interface distance, tissue interface position, tissue attenuation, tissue thickness, lesion spectral fingerprint). The monitoring unit is operable to provide feedback to a practitioner, such as graphical, audible, and/or haptic output of sensed data, and may also be operable to control operation of the at least one ablation element in response thereto.

### CROSS-REFERENCE TO RELATED APPLICATIONS

This application claims the benefit of U.S. provisional application No. 61/141,379, filed 30 Dec. 2008, which is hereby incorporated by reference as though fully set forth herein.

### BACKGROUND OF THE INVENTION

a. Field of the Invention

The instant disclosure relates to monitoring of diagnostic and therapeutic procedures. In particular, this disclosure relates to apparatus and methods for acoustically monitoring diagnostic and therapeutic procedures, for example cardiac ablation procedures utilized in the treatment of cardiac arrhythmia.

b. Background Art

It is well known that atrial fibrillation results from disorganized electrical activity in the heart muscle (the myocardium). The surgical maze procedure has been developed for treating atrial fibrillation, and involves the creation of a series of surgical incisions through the atrial myocardium in a preselected pattern so as to create conductive corridors of viable tissue bounded by scar tissue.

As an alternative to the surgical incisions of the maze procedure, transmural ablations of the heart may be used. Such ablations may be performed from within the chambers of the heart (endocardial ablation), using endovascular devices (e.g., catheters) introduced through arteries or veins. Various ablation techniques may be used, including, but not limited to, cryogenic ablation, radiofrequency ablation, laser ablation, ultrasonic ablation, and microwave ablation. The ablation devices are used to create elongated transmural lesions—that is, lesions extending through a sufficient thickness of the myocardium to block electrical conduction—forming the boundaries of the conductive corridors in the atrial myocardium. Perhaps most advantageous about the use of transmural ablation rather than surgical incision is the ability to perform ablation procedures without first establishing cardiopulmonary bypass (CPB).

It is desirable for the practitioner (e.g., the doctor or electrophysiologist) to be able to monitor various diagnostic and/or therapeutic parameters during an ablation procedure. Unfortunately, extant ablation devices often do not offer such feedback to the practitioner.

### BRIEF SUMMARY OF THE INVENTION

It is therefore an object of the invention to provide an ablation device that provides feedback to the practitioner about diagnostic and/or therapeutic parameters of interest, such as lesion making progress, tissue pre-pop detection, and tissue interface detection.

In one aspect, the present invention provides a medical device for ablating tissue including: an elongate catheter body having a tip region; at least one radiofrequency electrode located within the tip region of the elongate catheter body; and at least one acoustic transducer located within the tip region of the elongate catheter body to receive acoustic signals from proximate the tip region of the elongate catheter body. In some embodiments, the at least one radiofrequency electrode forms a tip of the elongate catheter body and the at least one acoustic transducer is positioned proximally adjacent the tip of the elongate catheter body. In other embodiments, the at least one acoustic transducer is positioned distally of the at least one radiofrequency electrode. In still other embodiments, the at least one acoustic transducer and the at least one radiofrequency electrode are co-located. For example, the at least one radiofrequency electrode may overly the at least one acoustic transducer and include an acoustically transparent thin metal electrode.

The at least one acoustic transducer may include at least one directional acoustic transducer. In these embodiments of the invention, the at least one acoustic transducer may be rotatable relative to at least a portion of the elongate catheter body. Alternatively, the tip region of the elongate catheter body may be rotatable relative to at least a portion of a remainder of the elongate catheter body. Of course, it is also contemplated that the at least one acoustic transducer may include at least one omnidirectional acoustic transducer, such as an annular, ring-shaped, or arc-shaped acoustic transducer, which may optionally include a flexible, wrappable piezopolymer.

The at least one acoustic transducer may include at least one passive acoustic transducer. It may also include at least one active acoustic transducer, such as a pinging or pulse-echo transducer. It is also desirable for the at least one acoustic transducer to have a frequency bandwidth of one of the following: at least about 50%, at least about 100%, and greater than 100%.

In some embodiments, the at least one acoustic transducer includes at least one of an acoustic matching layer and an acoustic lens. The acoustic lens may, for example, direct an acoustic beam at an angle to a longitudinal axis of the elongate catheter body, providing the at least one acoustic transducer with a field of view including a central region of a target tissue (e.g., to observe a forming lesion).

Also disclosed herein is a system for ablating tissue. The system includes an ablation catheter including: an elongate catheter body having a tip region; at least one radiofrequency electrode located within the tip region of the elongate catheter body; and at least one acoustic transducer located within the tip region of the elongate catheter body and operable to receive acoustic signals from proximate the tip region of the elongate catheter body.

The system also includes a monitoring unit coupled to the at least one acoustic transducer and operable to interpret the received acoustic signals as data regarding at least one therapeutic parameter. The monitoring unit typically includes at least one of a display and a speaker, such that the monitoring unit is operable to provide at least one of a graphical output and an audible output of the data regarding the at least one therapeutic parameter. Optionally, the monitoring unit is further operable to control operation of the at least one radiofrequency electrode responsive to the data regarding the at least one therapeutic parameter (e.g., to increase, decrease, or stop ablation).

In yet another aspect, the present invention provides a method of monitoring a tissue ablation procedure, including the following steps: providing an ablation catheter having a tip region including at least one radiofrequency electrode and at least one acoustic transducer; placing the ablation catheter adjacent a tissue to be ablated; delivering radiofrequency energy to the tissue to be ablated via the at least one radiofrequency electrode; and receiving at least one acoustic signal from proximate the tip region of the ablation catheter via the at least one acoustic transducer. Operation of the at least one transducer to receive the at least one acoustic signal may be gated by a power state of the at least one radiofrequency electrode.

As discussed herein, a passive transducer receives the sounds created by ablation naturally, while an active (e.g., pinging) transducer may receive both reflections of its transmitted pings and natural ablative sounds.

In the case of an active transducer (e.g., a pinging transducer), an electrical pulser may be utilized to drive the transducer. The pulser may optionally be included in the monitoring unit. Similarly, a receiver, used to detect incoming acoustic waves, may optionally be included in the monitoring unit.

The method optionally includes the following steps: sensing contact between the at least one radiofrequency electrode and a tissue to be ablated via the at least one acoustic transducer; sensing a thickness of the tissue to be ablated via the at least one acoustic transducer; monitoring creation of a lesion in the tissue to be ablated via the at least one acoustic transducer; and monitoring for pre-pop conditions in the tissue to be ablated via the at least one acoustic transducer.

The at least one acoustic signal may be interpreted as data regarding at least one, therapeutic parameter, and, in some embodiments, at least two therapeutic parameters. These therapeutic parameters may be selected from the group consisting of pre-pop detection, lesion making progress, tissue interface detection, tissue contact force, tissue contact establishment, bubble spatial distribution, bubble depth, bubble size, bubble size distribution, tissue interface distance, tissue interface position, tissue attenuation, tissue thickness, lesion spectral fingerprint, and changes in any of the foregoing.

Advantageously, catheters according to the present invention acoustically monitor diagnostic and/or therapeutic parameters of interest. This facilitates the collection and presentation of advisory data to the practitioner. It also permits closed-loop feedback control of a lesioning process. For example, tissue thickness is a therapeutic parameter insofar as it limits what ablation lesions can be formed.

The foregoing and other aspects, features, details, utilities, and advantages of the present invention will be apparent from reading the following description and claims, and from reviewing the accompanying drawings.

### BRIEF DESCRIPTION OF THE DRAWINGS

FIG. 1 is a perspective view of a catheter according to an embodiment of the present invention.

FIG. 2 depicts a catheter tip portion according to a first aspect of the present invention.

FIG. 3 depicts a catheter tip portion according to a second aspect of the present invention.

FIG. 4 depicts a catheter tip portion according to a third aspect of the present invention.

FIG. 5 depicts a catheter tip portion according to a fourth aspect of the present invention.

FIG. 6 depicts a catheter tip portion according to a fifth aspect of the present invention.

FIG. 7 depicts a catheter tip portion according to a sixth aspect of the present invention.

FIG. 8 depicts a handheld monitoring device according to some aspects of the present invention.

### DETAILED DESCRIPTION OF THE INVENTION

The present invention provides an ablation catheter incorporating acoustic monitoring of therapeutic and/or diagnostic parameters. For purposes of description, the present invention will be described and illustrated in connection with a radiofrequency (“RF”) ablation catheter, such as the LIVEWIRE™ steerable catheters and/or the LIVEWIRE TC™ ablation catheters of St. Jude Medical, Atrial Fibrillation Division, Inc. It is contemplated, however, that the described features and methods may be incorporated into any number of catheters or other devices, as would be appreciated by one of ordinary skill in the art.

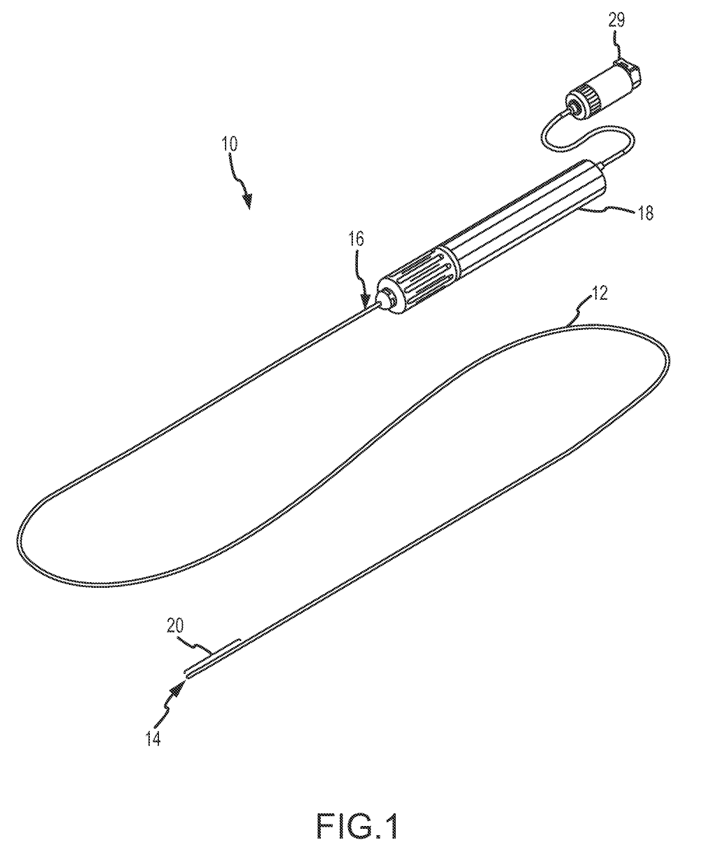

Referring now to the figures, and in particular to FIG. 1, an electrophysiology catheter 10 includes an elongate catheter body 12 having a distal end 14 and a proximal end 16. Catheter body 12 is typically flexible in order to be navigable through a patient's vasculature to an intended destination for diagnosis and/or therapy, such as introduction into a particular chamber of the patient's heart in order to ablate cardiac tissue during treatment of cardiac arrhythmia. Accordingly, a handle 18 may be coupled to proximal end 16 of catheter body 12 to control catheter 10. It is also desirable for at least the under-blood portion of catheter 10 to be disposable.

Distal end 14 of catheter body 12 includes tip region 20 thereof. As discussed in greater detail below, tip region 20 generally includes one or more RF ablating electrodes, such as tip and/or ring electrodes, and at least one acoustic transducer. As used herein, the term “tip region” refers to the distal-most segment of catheter body 12. Tip region 20 may also include sensing and/or pacing electrodes.

In some aspects of the invention, catheter body 12 may be deflected from a generally straight configuration into one or more curved or shaped configurations, including, without limitation, ring shapes, loop shapes, lasso shapes, and curvilinear arcs. Deflectability (or “steerability”) may be provided by one or more steering wires or pull wires (not shown) extending through catheter body 12 and connected to corresponding actuators (not shown) on handle 18 as generally known in the art. Typically, catheter 10 will be inserted into a patient in a generally straight configuration. By selectively deflecting catheter 10 using the actuators on handle 18, a physician can navigate distal end 14 thereof through the patient's vasculature to a desired location for treatment or diagnosis.

In other aspects of the invention, one or more shaping wires (e.g., one or more shape memory wires) are utilized to predispose one or more portions of catheter body 12 to assume a desired shape. For example, one or more shaping wires may be incorporated into tip region 20 such that tip region 20 is biased to assume a curved or other desirable shape. To straighten catheter body 12 for introduction into the patient's vasculature, one or more guiding introducers or sheaths may be used to position distal end 14 of catheter 10 at a desired location for treatment or diagnosis. That is, when catheter 10 is inserted into the guiding introducer or sheath, the guiding introducer or sheath will deform catheter 10 into a generally straight insertion configuration. When tip region 20 emerges from the end of the guiding introducer or sheath, the shaping wires will cause catheter 10 to resume its preselected shape. Alternatively, catheter 10 may be inserted through the working lumen of an intracardiac echocardiography (“ICE”) catheter or other lumenal medical device.

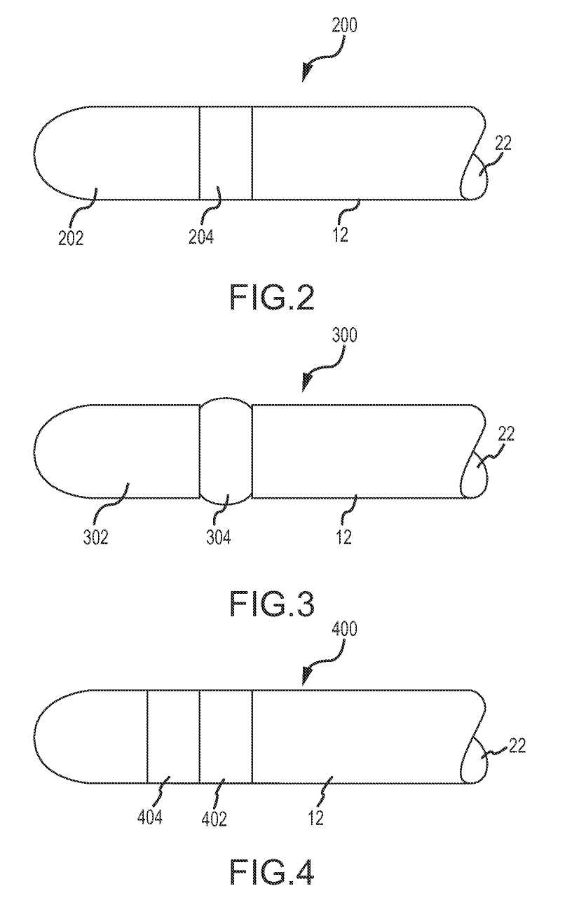

Typically, catheter body 12 will define at least one lumen 22 (e.g., FIG. 2) extending at least partially therethrough. As generally known in the art, lumen 22 may be employed, for example, to deliver an irrigating fluid to tip region 20 from an irrigation reservoir or to route one or more electrical leads (e.g., electrode and/or transducer ground and/or hot leads) between tip region 20 and proximal end 16. Irrigation may advantageously maintain the acoustic transducer(s) carried by tip region 20 at an acceptable temperature in order to avoid thermal damage thereto. Irrigation may also be used to cool an RF ablation tip electrode (such as electrodes 202 and 302, shown in FIGS. 2 and 3, respectively) in order to prevent charring and/or boiling.

One or more of the lumens may optionally extend through the wall of catheter body 12 so as to form one or more working ports. Such working ports may be used, for example, to introduce a fluid or gas, to introduce another medical device, to introduce a drug, or to allow catheter 10 to be advanced through the patient's vasculature over a guidewire. In the latter case, the working port may be regarded as the terminus of a guidewire lumen.

A connector plug 29 (FIG. 1) may be provided to couple catheter 10 to any hardware or systems desirable in connection with a particular diagnostic or therapeutic application of catheter 10. For example, plug 29 may couple catheter 10 to a localization system (e.g., the EnSite NavX™ navigation and visualization system of St. Jude Medical, Atrial Fibrillation Division, Inc.) to enable the position and/or orientation of tip region 20 within a patient to be measured and/or derived. Likewise, plug 29 may couple catheter 10 to one or more power supplies, such as an RF source to drive the RF ablation electrodes within tip region 20, and/or to a transducer pinger and/or receiver. In other embodiments of the invention, plug 29 couples catheter 10 to a handheld monitoring device of the type discussed in greater detail below. It is also contemplated that catheter 10 may be wirelessly coupled to any such hardware or systems or coupled directly to such systems without the use of plug 29.

Catheter 10 may also optionally incorporate other features of RF ablation catheters as generally known in the art. For example, one or more temperature sensors (e.g., thermocouples, thermistors, and/or temperature sensing optical fiber tips) may be provided within tip region 20 to measure a temperature at or near tip region 20. As another example, one or more spatial localization elements may be provided within tip region 20 to enable the position and/or orientation of tip region 20 within a patient to be measured and/or derived. In still other embodiments of the invention, tip region 20 also includes mapping and/or pacing electrodes to measure electrophysiological information from the surface of the heart, to pace the heart, and/or for any other desirable diagnostic or therapeutic purpose as generally known in the art. One of ordinary skill in the art will appreciate, therefore, that catheter 10 may incorporate a number of features in a variety of combinations consistent with the present invention.

FIGS. 2 through 7 are close-up views of the tip region of catheter body 12 according to several aspects of the invention. As described above, the tip region of catheter 10 generally includes at least one ablation element, such as a radiofrequency (“RF”) electrode, and at least one acoustic transducer. Of course, these numbers are merely exemplary, and one of ordinary skill in the art will appreciate that multiple ablation elements and/or multiple transducers may be employed consistent with the present teachings. For example, one may employ circumferentially-looking tubular transducers and/or forward looking disc transducers. One of ordinary skill in the art will also appreciate that alternative ablating elements, such as microwave ablating elements, high intensity ultrasound (“HIFU”) ablating elements, laser ablating elements, and the like are also suitable for use in connection with the present teachings as an alternative or addition to an RF ablating element.

The acoustic transducer is operable at least to receive incoming acoustic signals proximate the tip region of catheter body 12. In other aspects of the invention, the acoustic transducer is capable of both transmitting and receiving (e.g., pinging or operating in pulse-echo mode). Thus, the acoustic transducer will typically have a relatively acoustically unobstructed view of the diagnosis or therapy site proximate the tip region of catheter body 12 (e.g., a relatively acoustically unobstructed view of an actual or potential lesion site).

As discussed in detail below, the acoustic signals received by the acoustic transducer are interpreted as information concerning one or more therapeutic and/or diagnostic parameters. For example, a passive acoustic transducer (e.g., a transducer capable only of receiving acoustic signals) may be employed to receive natural boiling, bubbling, and/or cavitation sounds associated with tissue ablation. As another example, an active acoustic transducer (e.g., a transducer capable of both emitting and receiving acoustic signals, such as a pinging or pulse-echo transducer) may utilize the echoes of the signal it emits as an alternative to, or in addition to, tissue-produced acoustic signals emanating from the tissue being treated.

One of ordinary skill in the art will appreciate that a number of different types and constructions of acoustic transducers may be employed to good advantage in connection with the present invention. For example, the transducer or transducing material may be a piezoceramic, a piezopolymer, or a microelectromechanical (“MEMs”) based device such as a capacitive micromechanical ultrasonic transducer (“CMUT”). It may also be an electrostrictive material, a transducer responsive to pulsed optical illumination of tissue (e.g., photoacoustic imaging), an electrostatic membrane, or a magnetostrictive material.

Similarly, the acoustic transducers utilized in connection with the present invention may include one addressable element or an array of several addressable elements. Likewise, the acoustic transducers may be mechanically focused (e.g., focused through shaping or the use of an acoustic lens) or electronically focused. Of course, the acoustic transducers may also include acoustic matching layers and/or attenuative backing materials as generally known in the art.

As described above, it is contemplated that the acoustic transducer may operate either passively (e.g., may only be capable of receiving acoustic signals) or actively (e.g., may be capable of both emitting and receiving acoustic signals, such as in a pinging or pulse-echo mode). It is also contemplated that the acoustic transducer may be either directional (e.g., capable of emitting and/or receiving acoustic signals along only a single direction or subset of directions) or omnidirectional (e.g., capable of emitting and/or receiving acoustic signals from a wide range of directions, such as about 360 degrees). Advantageously, the use of an omnidirectional acoustic transducer eliminates the need to rotate the transducer relative to the tissue being observed.

It is desirable for the acoustic transducer to have a high resonant frequency and a wide bandwidth in order to provide fine range-resolution and to facilitate detection of both high frequency content and low frequency content (e.g., boiling and spattering). Advantageously, the acoustic transducer has a frequency bandwidth of at least about 50%, preferably close to about 100%, and most preferably of over about 100%. For larger bandwidths, it is desirable to include one or more acoustic matching layers coupled to the acoustic transducer.

Several embodiments of the tip region of catheter 10, exemplary of different types and constructions of the ablation element and the acoustic transducer, are depicted in FIGS. 2-7. The ordinary artisan will understand from this disclosure how to select and arrange one or more ablation elements and one or more acoustic transducers for a particular application of a catheter 10 according to the present invention.

FIG. 2 depicts a tip region 200 according to a first aspect of the present invention. As shown in FIG. 2, tip region 200 includes an RF electrode 202 that forms the tip of catheter body 12. An acoustic transducer 204, which may be either active or passive, is positioned proximally of and adjacent to RF electrode 202. Acoustic transducer 204 is an annular (e.g., ring- or arc-shaped) omnidirectional transducer that provides an acoustic field of view about the circumference of tip region 200, thereby eliminating the need to rotate the catheter about its long axis.

Acoustic transducer 204 may be formed from a wrappable, flexible piezopolymer (e.g., PVDF, PVDF-TRE, nylon) and bonded to catheter body 12 at the indicated location, such as by application of a suitable adhesive and/or thermal energy to acoustic transducer 204 and/or catheter body 12. Alternatively, acoustic transducer 204 may take the form of a piezoceramic ring (e.g., monolithic PZT) installed on catheter body 12 at the indicated location. In still other embodiments of the invention, acoustic transducer 204 incorporates a conforming piezocomposite (e.g., polymer/PZT mat). The transducer 204 may also include a platinum, platinum-iridium, iridium, or gold film or surface coating on its blood-contacting surface. Such a coating advantageously allows the transducer 204 to act as part of RF electrode 202 during ablation.

Typically, acoustic transducer 204 includes a pair of thin-film electrodes on its internal and external diameters, such that sensed acoustic signals create voltage variations across these electrodes and such that acoustic transducer 204, if active, can be pinged by an applied voltage pulse across its thickness. A suitable receive amplifier, which may be incorporated into catheter 10 or attached externally thereto (e.g., via plug 29) can detect the voltage variations.

FIG. 3 depicts a tip region 300 according to a second aspect of the present invention. As shown in FIG. 3, tip region 300 includes an RF electrode 302 that forms the tip of catheter body 12. An acoustic transducer 304, which may be either active or passive, is positioned proximally of and adjacent to RF electrode 302. As with acoustic transducer 204 illustrated in FIG. 2 and discussed above, acoustic transducer 304 is an annular (e.g., ring- or arc-shaped) omnidirectional transducer that provides an acoustic field of view around the circumference of tip region 300. In contrast to acoustic transducer 204, acoustic transducer 304 has a curved surface. This curved surface functions as an acoustic lens that focuses or defocuses the outgoing and/or incoming acoustic signals. For example, acoustic transducer 304 could have a convex curve (as depicted in FIG. 3) epoxy lens that serves to broaden the directivity of the transducer in the planes containing the catheter longitudinal axis. Alternatively, the lens may be designed to cause the directivity to be tilted “forward” towards the tip of the catheter, allowing assessment of tissue regions laterally away from the transducer face and more in front of RF electrode 302.

FIG. 4 depicts a tip region 400 according to a third aspect of the present invention. As shown in FIG. 4, tip region 400 includes an RF ring electrode 402 positioned proximally of the tip of catheter body 12. An omnidirectional annular acoustic transducer 404, which may be either active or passive, is positioned distally of RF ring electrode 402. Thus, FIGS. 2-4 illustrate that the acoustic transducer may be positioned either proximally or distally of the RF electrode.

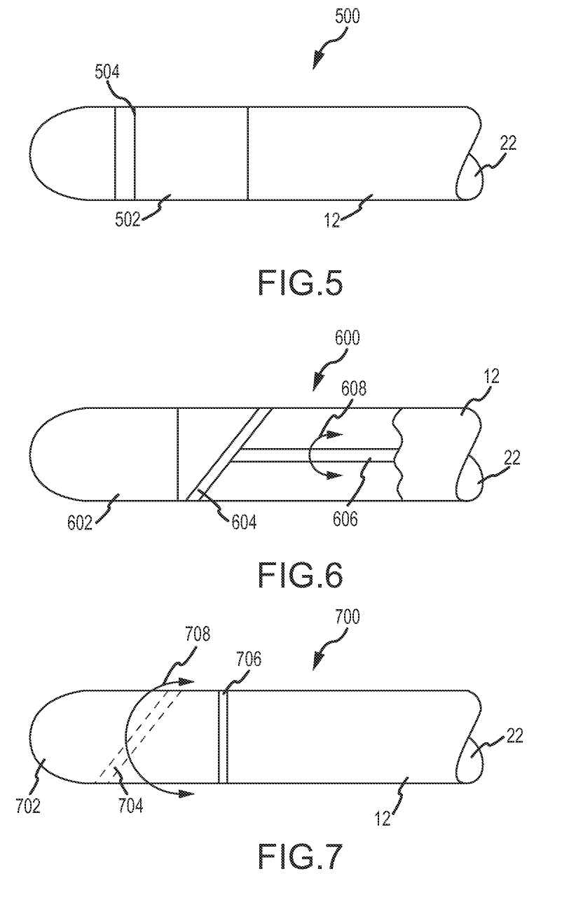

FIG. 5 depicts a tip region 500 according to a fourth aspect of the present invention. As shown in FIG. 5, tip region 500 includes an RF electrode 502 that forms the tip of catheter body 12. An acoustic transducer 504, which may be either active or passive, is co-located with RF electrode 502. That is, acoustic transducer 504 and RF electrode 502 overlap rather than having a distal/proximal relationship. As shown in FIG. 5, acoustic transducer 504 is disposed upon RF electrode 502, but it is contemplated that acoustic transducer 504 could optionally be integrated “inside” RF electrode 502. That is, acoustic transducer 504 could be positioned inside of lumen 22 beneath RF electrode 502 without departing from the spirit and scope of the present invention.

While RF electrode 502 should be electrically conductive, this does not imply that it must be metallic. Indeed, RF electrode 502 may be a carbon-based conductive electrode.

Note that in this embodiment, the transducer 504 can be located in the “middle” of the RF electrode 502. Advantageously, the transducer outer surface may also act as an RF ablation electrode in addition to acting as an acoustic window, such as by coating the transducer 504 with an appropriate film as described above. In this manner, transducer 504 looks straight into the RF lesion, advantageously without requiring any beam steering (e.g., via an acoustic lens).

FIG. 6 depicts a tip region 600 according to a fifth aspect of the present invention. As shown in FIG. 6, tip region 600 includes an RF electrode 602 and a disc-shaped acoustic transducer 604. Disc-shaped acoustic transducer 604, which may be either active or passive, is directional, and therefore provides an acoustic field of view along only one direction rather than about the circumference of tip region 600. To provide a larger acoustic field of view, disc-shaped acoustic transducer 604 is mounted (e.g., about a shaft 606) to be rotatable relative to at least a portion of catheter body 12 as indicated by arrows 608. For example, acoustic transducer 604 may be motorized or manually rotated as generally known in the art. Alternatively, the impeller-based assembly disclosed in U.S. application Ser. No. 12/347,116, which is hereby incorporated by reference as though fully set forth herein, could be advantageously employed in connection with the present invention in order to cause acoustic transducer 604 to rotate. It should be understood that the cylindrical containment of acoustic transducer 604 is desirably acoustically transparent.

FIG. 7 depicts a tip region 700 according to a sixth aspect of the invention. As shown in FIG. 7, tip region 700 includes an RF electrode 702 and a disc-shaped directional active or passive acoustic transducer 704, shown in phantom, co-located with RF electrode 702. In contrast to tip region 600 illustrated in FIG. 6, where acoustic transducer 604 rotates relative to a stationary RF electrode 602 forming the tip of catheter body 12, in the embodiment illustrated in FIG. 7, the tip assembly, including both RF electrode 702 and acoustic transducer 704, rotates relative to the remainder of catheter body 12. The tip assembly is joined to catheter body 12 via a rotating joint or interface assembly 706 and driven by a gearing arrangement or torque wire, that permits the entire tip assembly to rotate relative to catheter body 12 as indicated by arrows 708. As with the embodiment of FIG. 6, one of ordinary skill in the art will appreciate the desirability of making RF electrode 702 both electrically conductive and acoustically transparent, for example by employing a thin film metal overlaid on an acoustically transparent polymer.

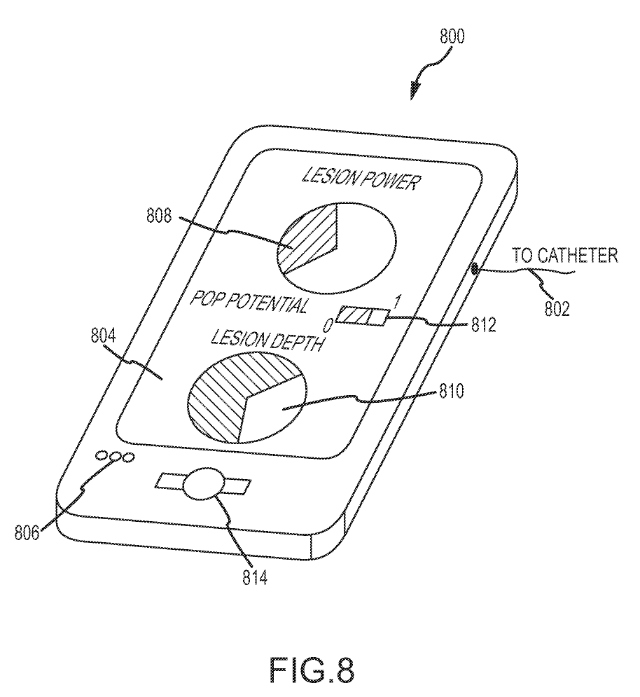

In use, catheter 10 may be coupled to a monitoring unit, such as the handheld monitoring unit 800 depicted in FIG. 8. In some embodiments of the invention, catheter 10 may be coupled to monitoring unit 800 via a wire 802, though it is contemplated that catheter 10 may also communicate wirelessly with monitoring unit 800. In general, monitoring unit 800 (or another suitable monitoring unit) is coupled to the acoustic transducer and operable to interpret acoustic signals received by the acoustic transducer as data regarding at least one diagnostic or therapeutic parameter. This data regarding at least one diagnostic or therapeutic parameter is referred to herein as “sensed data.”

For example, in some embodiments of the invention, the sensed data is utilized to detect, and optionally warn of, pre-pop conditions in the tissue being ablated. The pre-popping state will have a distinct acoustic signature relating to the exponential increase in bubble coalescence and tissue microrupture between such bubbles. By monitoring for this distinct acoustic signature, a practitioner can be warned of impending tissue popping, allowing the practitioner to cease ablation, reduce power, or take other action to mitigate the risk of tissue popping. Alternatively, monitoring unit 800 may be configured to automatically reduce, cut off, or otherwise control the operation of the RF electrode upon detecting a pre-pop condition.

In other embodiments of the invention, the sensed data indicates lesion making progress. For example, passive acoustic transducers may be employed to listen to the overall acoustic din or spectrum of lesion making progress. Such noise may optionally be power-integrated, and is proportional to lesion volume. Similarly, active acoustic transducers may be used to ping and observe “quiet” features such as stagnant bubbles and their distances and depth distributions or to monitor cavitation events. Sensed data could also include the acoustic reflectivity of a targeted tissue location or interface. Such data could optionally be mapped to provide a two-dimensional (e.g., areal) or three-dimensional (e.g., volumetric) image of the targeted tissue.

In still other embodiments of the invention, sensed data is representative of the position of one or more tissue interfaces (e.g., the location of the esophagus, aorta, and lungs), thereby allowing the calculation of tissue thicknesses and facilitating the creation of tissue maps. One suitable method for detecting tissue interfaces, measuring tissue thickness, and/or creating tissue maps using acoustic energy is described in U.S. application Ser. No. 12/533,307, filed 31 Jul. 2009, which is hereby incorporated by reference as though fully set forth herein.

Other diagnostic and therapeutic parameters that can be monitored acoustically, directly or indirectly, include, without limitation, tissue contact force, tissue contact establishment, bubble spatial distribution, bubble depth, bubble size, bubble size distribution, tissue interface distance, tissue interface position, tissue attenuation, and lesion spectral fingerprints. Monitoring unit 800 may also interpret acoustic signals received by the acoustic transducer as sensed data regarding a change in one or more of the diagnostic or therapeutic parameters being monitored.

Monitoring unit 800 may include a display 804 and/or a speaker 806 to provide graphical and/or audible output, respectively, of the sensed data regarding the therapeutic and/or diagnostic parameter(s) being monitored, as well as of other parameters related to the ablation process. For example, display 804 may provide a graphical representation 808 of lesion power, a graphical representation 810 of lesion depth, and/or a graphical representation 812 of pop potential. Likewise, speaker 806 may sound an audible warning to alert a practitioner to a pre-pop condition. Haptic feedback (e.g., causing monitoring unit 800 and/or catheter 10 to vibrate) is also contemplated as an alternative or addition to graphical and/or audible feedback. One suitable feedback indicator would include monitoring and displaying or annunciating (e.g., via a tone, synthetic speech, or the like) a rate of change of a sensed property, such as acoustic reflectivity, wherein the sensed property initially changes rapidly then asymptotically changes less and less as a lesion approaches its final size.

It should be understood that the sensed data may be provided to the practitioner as advisory data (e.g., graphical, audible, and/or haptic output). Alternatively or additionally, the sensed data may be used to automatically control the ablation procedure. Suitable controls (e.g., a touchscreen and/or buttons 814), which are preferably water-resistant, will typically also be provided to permit the practitioner to provide inputs to monitoring unit 800.

The sensed data may be normalized, scaled, or processed in other ways prior to being presented to the practitioner. For example, the sensed data may optionally be processed by comparing it to a patient population from a database, by fitting it to a model or curve stored in memory, or by utilizing a data processing algorithm customized for a particular catheter 10. The sensed data may also be tagged with an anatomical location, such as disclosed in U.S. application Ser. No. 12/533,307. Of course, the sensed data may also be stored in a suitable memory device for later retrieval and/or analysis.

According to some aspects of the invention, monitoring unit 800 interprets the acoustic signals received as sensed data regarding at least two diagnostic or therapeutic parameters. These two parameters can then be correlated or compared in order to corroborate the detection of a pre-pop condition, the progress of a lesion, or the like. Of course, the interpreted acoustic signals may be received by different acoustic transducers, received simultaneously by the same acoustic transducer, or received sequentially by the same acoustic transducer (e.g., by time-gating reception in order to distinguish between signals coming from further away, such as from a direction opposite the tissue-facing surface of the acoustic transducer). As an example, received signals at two different frequencies may be observed. As another example, one might receive an acoustic signal and electrically monitor the lesion site using the RF ablation tip as a sensing or pacing electrode when not in ablating mode.

In an exemplary procedure, catheter 10 is introduced into a patient's heart chamber via the vasculature. The tip region of catheter 10 is brought into contact with a tissue to be ablated. The at least one acoustic transducer carried on the tip region of catheter 10 can be used to sense contact between the tip region (e.g., the at least one RF electrode) and the tissue to be ablated; assess a pre-lesion tissue state; and/or sense a thickness of the tissue to be ablated. Tip contact is detectable because, as the tip is mechanically loaded, the transducer loading and resonances are affected reproducibly.

The at least one RF ablation electrode may then be activated to deliver RF energy to the tissue to be ablated. Advantageously, the at least one RF electrode may be driven with a waveform that is known to produce useful thermoacoustic or thermal-microbubbling acoustic signatures in tissue. While ablating the tissue, the at least one acoustic transducer may be used to monitor lesion progress and detect pre-pop conditions, as well as other diagnostic and/or therapeutic parameters. It is contemplated that operation of the at least one acoustic transducer may be gated by a power state of the at least one RF electrode or vice-versa (that is, operation of the at least one RF electrode may be gated by operation of the at least one acoustic transducer). Operation of the at least one acoustic transducer may also be constant during at least one power-state transition in RF power delivery or periodic.

Microbubbles have been found to form in any thermal ablation when the temperature is high enough to (a) cause gas dissolution from blood; and/or (b) cause boiling-based steam bubbles to form. Phenomenon (a) can happen even at relatively low temperatures, just as a glass of tap water evolves bubbles when placed on a table at room temperature. Phenomenon (b) generally takes place when tissue reaches temperatures approaching and exceeding 100° C.

Although several embodiments of this invention have been described above with a certain degree of particularity, those skilled in the art could make numerous alterations to the disclosed embodiments without departing from the spirit or scope of this invention. For example, it is contemplated that suitable electronics and/or power supplies for the RF electrodes and acoustic transducers described herein could be incorporated into one or more of handle 18 of catheter 10, plug 29 attached to catheter 10, a localization system, monitoring unit 800, an RF power supply, or even carried on board catheter 10 (e.g., incorporated into the tip region thereof). As another example, a practitioner could bodily rotate all of the catheter body so as to orient a directional acoustic transducer towards a tissue of interest rather than rotating the transducer relative to at least a portion of the catheter body (e.g., a rotating transducer or a rotating tip region).

All directional references (e.g., upper, lower, upward, downward, left, right, leftward, rightward, top, bottom, above, below, vertical, horizontal, clockwise, and counterclockwise) are only used for identification purposes to aid the reader's understanding of the present invention, and do not create limitations, particularly as to the position, orientation, or use of the invention. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other.

It is intended that all matter contained in the above description or shown in the accompanying drawings shall be interpreted as illustrative only and not limiting. Changes in detail or structure may be made without departing from the spirit of the invention as defined in the appended claims.

What is claimed is:

1. A medical device for ablating tissue, comprising:
an elongate catheter body having a tip region;
at least one radiofrequency electrode located within the tip region of the elongate catheter body; and
at least one acoustic transducer located within the tip region of the elongate catheter body to receive acoustic signals from proximate the tip region of the elongate catheter body.

2. The medical device according to claim 1, wherein the at least one radiofrequency electrode forms a tip of the elongate catheter body and the at least one acoustic transducer is positioned proximally adjacent the tip of the elongate catheter body.

3. The medical device according to claim 1, wherein the at least one acoustic transducer is positioned distally of the at least one radiofrequency electrode.

4. The medical device according to claim 1, wherein the at least one acoustic transducer and the at least one radiofrequency electrode are co-located.

5. The medical device according to claim 4, wherein the at least one radiofrequency electrode overlies the at least one acoustic transducer and comprises an acoustically transparent thin metal electrode.

6. The medical device according to claim 1, wherein the at least one acoustic transducer comprises at least one directional acoustic transducer.

7. The medical device according to claim 6, wherein the at least one acoustic transducer is rotatable relative to at least a portion of the elongate catheter body.

8. The medical device according to claim 7, wherein the tip region of the elongate catheter body is rotatable relative to at least a portion of a remainder of the elongate catheter body.

9. The medical device according to claim 1, wherein the at least one acoustic transducer comprises at least one omnidirectional acoustic transducer.

10. The medical device according to claim 9, wherein the at least one acoustic transducer comprises at least one annular, ring-shaped, or arc-shaped acoustic transducer.

11. The medical device according to claim 9, wherein the at least one acoustic transducer includes a flexible, wrappable piezopolymer.

12. The medical device according to claim 1, wherein the at least one acoustic transducer comprises at least one passive acoustic transducer.

13. The medical device according to claim 1, wherein the at least one acoustic transducer comprises at least one active pulse-echo acoustic transducer.

14. The medical device according to claim 1, wherein the at least one acoustic transducer has a frequency bandwidth of one of the following: at least about 50%, at least about 100%, and greater than 100%.

15. The medical device according to claim 1, wherein the at least one acoustic transducer includes at least one of an acoustic matching layer and an acoustic lens.

16. The medical device according to claim 15, wherein the acoustic lens directs an acoustic beam at an angle to a longitudinal axis of the elongate catheter body such that the at least one acoustic transducer has a field of view including a central region of a target tissue.

17. A system for ablating tissue, comprising:
an ablation catheter comprising:
an elongate catheter body having a tip region;
at least one radiofrequency electrode located within the tip region of the elongate catheter body; and
at least one acoustic transducer located within the tip region of the elongate catheter body and operable to receive acoustic signals from proximate the tip region of the elongate catheter body; and
a monitoring unit coupled to the at least one acoustic transducer and operable to interpret the received acoustic signals as data regarding at least one therapeutic parameter.

18. The system according to claim 17, wherein the monitoring unit includes at least one of a display and a speaker and the monitoring unit is operable to provide at least one of a graphical output and an audible output of the data regarding the at least one therapeutic parameter.

19. The system according to claim 17, wherein the monitoring unit is further operable to control operation of the at least one radiofrequency electrode responsive to the data regarding the at least one therapeutic parameter.

20. A method of monitoring a tissue ablation procedure, comprising:
providing an ablation catheter having a tip region including at least one radiofrequency electrode and at least one acoustic transducer;
placing the ablation catheter adjacent a tissue to be ablated;
delivering radiofrequency energy to the tissue to be ablated via the at least one radiofrequency electrode; and
receiving at least one acoustic signal from proximate the tip region of the ablation catheter via the at least one acoustic transducer.

21. The method according to claim 20, further comprising interpreting the at least one acoustic signal as data regarding at least one therapeutic parameter selected from the group consisting of pre-pop detection, lesion making progress, tissue interface detection, tissue contact force, tissue contact establishment, bubble spatial distribution, bubble depth, bubble size, bubble size distribution, tissue interface distance, tissue interface position, tissue attenuation, tissue thickness, lesion spectral fingerprint, and changes in any of the foregoing.

22. The method according to claim 21, wherein the step of interpreting the at least one acoustic signal as data regarding at least one therapeutic parameter comprises interpreting the at least one acoustic signal as data regarding two or more therapeutic parameters.

23. The method according to claim 20, wherein operation of the at least one transducer to receive the at least one acoustic signal is gated by a power state of the at least one radiofrequency electrode.

24. The method according to claim 20, further comprising:
sensing contact between the at least one radiofrequency electrode and a tissue to be ablated via the at least one acoustic transducer;
sensing a thickness of the tissue to be ablated via the at least one acoustic transducer;
monitoring creation of a lesion in the tissue to be ablated via the at least one acoustic transducer; and
monitoring for pre-pop conditions in the tissue to be ablated via the at least one acoustic transducer.
